(19) United States
(12) Patent Application Publication
(10) Pub. No.: US 2006/0029574 A1
(43) Pub. Date: Feb. 9, 2006

(54) Biomarkers for diagnosis, prognosis, monitoring, and treatment decisions for drug resistance and sensitivity

(75) Inventors: Maher Albitar, Coto De Caza, CA (US); Hagop Kantarjian, Bellaire, TX (US); Ira L. Goldknopf, The Woodlands, TX (US); Essam Sheta, The Woodlands, TX (US)

Correspondence Address: DAVID L. PARKER;FULBRIGHT & JAWORSKI L.L.P., 600 CONGRESS AVENUE, SUITE 2400, AUSTIN, TX, 78701, US

(73) Assignee: Board of Regents, The University of Texas System

(21) Appl. No.: 10/913,296

(22) Filed: Aug. 6, 2004

(51) Int. Cl. G01N 33/574

(52) U.S. Cl. 424/93.1; 435/7.23

(57) ABSTRACT

The present invention provides methods and compositions for identifying cancer cells that are either sensitive or resistant to a particular anti-cancer therapy. Accordingly, the present invention allows for more accurate diagnosis, prognosis, and monitoring of a subject's condition. Furthermore, the ability to assess a subject's resistance or sensitivity to a particular treatment regimen will permit more informed treatment decisions to be made prior to beginning therapy. The present invention also overcomes deficiencies in the prior art concerning the treatment of cancers by providing methods and compositions for treating cancer and improving the effectiveness of other cancer therapies.

### BACKGROUND OF THE INVENTION

A. Field of the Invention

The present invention relates generally to the field of cancer biology. More particularly, it concerns protein markers for diagnosis, prognosis, monitoring, and treatment decisions for drug resistance and sensitivity. In addition, the invention concerns methods and compositions for overcoming drug resistance and maximizing the effectiveness of anti-cancer therapies.

B. Description of Related Art

Cancer is the second leading cause of death in the United States. An estimated 563,700 Americans will die of cancer in 2004 (Cancer Facts and Figures. 2004, American Cancer Society). Although a number of anti-cancer agents are available for the treatment of cancer, cancer cell resistance to these agents remains a major problem in clinical oncology. One example is the small molecule Abl kinase inhibitor, imatinib mesylate (Gleevec®), which has shown dramatic success in treating chronic myelogenous leukemia (CML). Nevertheless, some patients are resistant to imatinib mesylate, and others who initially respond eventually relapse and progress on therapy.

It is unclear why some patients develop resistance to imatinib mesylate or other anti-cancer agents, and what can be done to prevent or delay the onset of resistance. With regard to imatinib mesylate, resistance has been associated with amplification or mutation of the BCR-ABL fusion gene (Shah et al., 2002; Gorre et al., 2001; Branford et al., 2002; Hochhaus et al., 2002). It has also been suggested that resistance to imatinib mesylate may be due to inactivation by binding to α-1 acid glycoprotein (Gambacorti-Passerini et al., 2000; Gambacorti-Passerini et al., 2002; Le Coutre et al., 2002). The overexpression of P-glycoprotein has also been implicated in imatinib mesylate resistance (Mahon et al., 2003). Cells may also become resistant to imatinib mesylate through the increased usage of signal transduction pathways that do not depend on the Bcr-Abl oncoprotein; however, these pathways remain undefined.

Previously, the ability to predict which patients are, or will become, resistant to a particular therapy has been limited. The ability to predict a patient's response to therapy would be a valuable asset in developing treatment strategies. For example, a patient who is identified as being resistant to imatinib mesylate could be treated with an alternate therapy or with more aggressive imatinib mesylate therapy (e.g., higher dosage and/or in combination with other therapeutic agents).

Although reliable individual diagnostic, prognostic, and predictive tools are limited at present, proteomics may provide new indicators and drug targets for malignancies. Proteomics has previously been used in the study of leukemia. For example, two-dimensional polyacrylamide gel electrophoresis (2-D PAGE) of proteins from the lymphoblasts of patients with ALL was used to identify polypeptides that could distinguish between the major subgroups of ALL (Hanash et al., 1986). In other studies of ALL using 2-D PAGE, distinct levels of a polypeptide were observed between infants and older children with otherwise similar cell surface markers (Hanash et al., 1989). Voss et al. demonstrated that B-CLL patient populations with shorter survival times exhibited changed levels of redox enzymes, Hsp27, and protein disulfide isomerase, as determined by 2-D PAGE of proteins prepared from mononuclear cells (Voss et al., 2001). As these studies indicate, proteomics can be a useful tool in the study of cancer.

There remains a need for improved methods and compositions for identifying patients who are resistant, or are likely to develop resistance, to a particular cancer therapy. Additionally, there is a need for improved methods and compositions for the treatment of drug-resistant cancers.

### SUMMARY OF THE INVENTION

The present invention overcomes the deficiencies in the prior art by providing methods and compositions for identifying cancer cells that are either sensitive or resistant to a particular anti-cancer therapy. Accordingly, the present invention allows for more accurate diagnosis, prognosis, and monitoring of a subject's condition. Furthermore, the ability to assess a subject's resistance or sensitivity to a particular treatment regimen will permit more informed treatment decisions to be made at the onset of therapy. In addition, the present invention overcomes deficiencies in the prior art concerning the treatment of cancers by providing methods and compositions for treating cancer and improving the effectiveness of other cancer therapies.

In one embodiment, the present invention provides a method for identifying a protein, a group of proteins, or a protein pattern associated with sensitivity or resistance to an anti-cancer agent comprising: obtaining a first cell, wherein the first cell is sensitive to the anti-cancer agent; obtaining a second cell, wherein the second cell is resistant to the anti-cancer agent; and identifying a protein, a group of proteins, or a protein pattern that is differentially expressed between the first cell and the second cell, wherein the differentially expressed protein, group of proteins, or protein pattern is associated with sensitivity or resistance to the anti-cancer agent.

Any type of cell may be used in the method for identifying a protein, a group of proteins, or a protein pattern associated with sensitivity or resistance to an anti-cancer agent, so long as the cell can be characterized as either resistant or sensitive to the particular anti-cancer agent. Resistance or sensitivity may be assessed on laboratory-based or clinical criteria. Furthermore, resistance may be primary, where resistance is identified in a cell line or subject that has not previously been exposed to the anti-cancer agent, or secondary, in a situation where resistance occurs after an initial response to the anti-cancer agent.

In certain aspects of the invention the first cell is obtained from a first subject and the second cell is obtained from a second subject. In one embodiment, the first subject and the second subject have cancer.

In one embodiment, the present invention provides a method for identifying a protein, a group of proteins, or a protein pattern associated with sensitivity or resistance to an Abl kinase inhibitor comprising: obtaining a first cell, wherein the first cell is sensitive to the Abl kinase inhibitor; obtaining a second cell, wherein the second cell is resistant to the Abl kinase inhibitor; and identifying a protein, a group of proteins, or a protein pattern that is differentially expressed between the first cell and the second cell, wherein the differentially expressed protein, group of proteins, or protein pattern is associated with sensitivity or resistance to the Abl kinase inhibitor.

It is contemplated that the methods of the present invention may be used to identify a protein, a group of proteins, or a protein pattern associated with sensitivity or resistance to any Abl kinase inhibitor. Those of skill in the art are familiar with Abl kinase inhibitors. Non-limiting examples of Abl kinase inhibitors include: BMS354825, and pyrido[3,5-d]pyrimadines such as PD173955 and PD166326. In a preferred embodiment, the Abl kinase inhibitor is imatinib mesylate.

Any type of cell may be used in the method for identifying a protein, a group of proteins, or a protein pattern associated with sensitivity or resistance to an Abl kinase inhibitor, so long as the cell can be characterized as either resistant or sensitive to the particular Abl kinase inhibitor. Resistance or sensitivity may be assessed on laboratory-based or clinical criteria. Furthermore, resistance may be primary, where resistance is identified in a cell line or subject that has not previously been exposed to the Abl kinase inhibitor, or secondary, in a situation where resistance occurs after an initial response to the Abl kinase inhibitor.

Those of skill in the art will be familiar with methods and criteria for characterizing a cell or a subject as sensitive or resistant to an Abl kinase inhibitor. For example, a cell may be considered sensitive to a particular Abl kinase inhibitor if it is obtained from a subject who demonstrates sensitivity to the Abl kinase inhibitor. A cell may be considered resistant to a particular Abl kinase inhibitor if it is obtained from a subject who demonstrates resistance to the Abl kinase inhibitor. The cell may be obtained from the subject before, during, or after treatment. Criteria for evaluating a subject's response to an Abl kinase inhibitor may be defined at the hematologic or cytogenetic level. A subject may be regarded as having a hematologic response if he has achieved normal leukocyte and platelet levels within three months of starting Abl kinase inhibitor treatment. A subject may be regarded as having a cytogenetic response if within twelve months of starting Abl kinase inhibitor treatment no Philadelphia-chromosome positive cells are observed on examination of 30 bone marrow metaphases. A subject may be regarded as a potential responder if after three months of Abl kinase inhibitor treatment he has achieved a cytogenetic response of greater than 35% Philadelphia-chromosome negative metaphases, and thereafter he continues to achieve a further 30 point or more reduction in the percentage of Philadelphia-chromosome positive metaphases at each three month interval.

Another method for identifying a cell as resistant to an Abl kinase inhibitor is if the cell is capable of surviving culturing with the inhibitor for 48 hours. A cell may be considered sensitive to an Abl kinase inhibitor if it dies upon culturing with the inhibitor for 48 hours.

In certain aspects of the invention the first cell is obtained from a first subject and the second cell is obtained from a second subject. In one embodiment, the first subject and the second subject have cancer. The cancer may be any cancer that is treatable with an Abl kinase inhibitor, such as cancers associated with activated ABL, KIT, PDGFR, ARG, or other kinases found to be inhibited by Abl kinase inhibitors. In some embodiments the cancer is leukemia, gastrointestinal stromal tumor, systemic mastocytosis, hyperesosinophilic syndrome, or other myeloproleferative diseases. In some embodiments the cancer is breast cancer, soft tissue sarcoma, ovarian cancer, pelvic cancer, or peritoneal cancer. In one embodiment, the first subject and the second subject have leukemia. The leukemia may be chronic myelogenous leukemia (CML), acute myelogenous leukemia (AML), chronic lymphocytic leukemia (CLL), or acute lymphocytic leukemia (ALL). In another embodiment, the first subject and the second subject have a gastrointestinal stromal tumor.

In some embodiments, identifying the protein, the group of proteins, or the protein pattern involves performing two-dimensional gel electrophoresis. Two-dimensional gel electrophoresis is well known to those of skill in the art, and has been described in, for example, U.S. Pat. Nos. 5,534,121 and 6,398,933, both of which are incorporated herein by reference.

In certain embodiments, identifying the protein, the group of proteins, or the protein pattern involves performing mass spectrometry. Those of skill in the art are familiar with the use of mass spectrometry in the identification of proteins. In some embodiments, the mass spectrometry is matrix-assisted laser desorption ionization time-of-flight mass spectrometry (MALDI-TOF MS), surface-enhanced laser desorption ionization time-of-flight mass spectrometry (SELDI-TOF MS), or tandem mass spectrometry (MS-MS).

In one embodiment, the present invention provides a method for predicting a subject's sensitivity or resistance to an Abl kinase inhibitor comprising: obtaining a sample from the subject; determining a protein expression profile for the subject; and comparing the subject's protein expression profile with a reference protein expression profile to predict the subject's sensitivity or resistance to an Abl kinase inhibitor. The protein expression profile may be determined by a variety of approaches. For example, the protein expression profile may be determined by evaluating protein levels or by evaluating transcription levels.

In some embodiments, determining the protein expression profile involves performing two-dimensional gel electrophoresis. In some embodiments, determining the protein expression profile involves performing mass spectrometry. In certain embodiments, determining the protein expression profile involves performing both two-dimensional gel electrophoresis and mass spectrometry. In yet other embodiments, determining the protein expression profile involves performing RT-PCR.

The protein expression profile may comprise one or more proteins or protein markers. In one embodiment, the protein expression profile comprises one or more of the proteins in Table 1. All of the Accession Numbers listed in Table 1 are incorporated herein by reference.

In another embodiment, the present invention provides a method of predicting response to therapy in a patient with cancer comprising: obtaining a sample from the patient; and evaluating the expression of one or more of the proteins listed in Table 1 in the patient's sample to predict the patient's response to therapy. In some embodiments, the method further comprises comparing the expression of one or more of the proteins listed in Table 1 in the patient's sample with a reference sample associated with a known response to the therapy.

In certain aspects, the therapy is chemotherapy, radiotherapy, immune therapy, or gene therapy, or a combination of the above. In one embodiment, the chemotherapy is Abl kinase inhibitor therapy. In a preferred embodiment, the Abl kinase inhibitor therapy is imatinib mesylate therapy.

The patient may have any form of cancer. Examples of cancers include, but are not limited to, breast cancer, lung cancer, prostate cancer, ovarian cancer, brain cancer, liver cancer, cervical cancer, colon cancer, renal cancer, skin cancer, head & neck cancer, bone cancer, esophageal cancer, bladder cancer, uterine cancer, lymphatic cancer, stomach cancer, pancreatic cancer, testicular cancer, or leukemia. In one embodiment, the cancer is a hematologic malignancy. The hematologic malignancy may be leukemia, non-Hodgkin lymphoma, Hodgkin lymphoma, myeloma, or myelodysplastic syndrome. In certain aspects of the invention, the leukemia is acute myelogenous leukemia, chronic myelogenous leukemia, acute lymphocytic leukemia, or chronic lymphocytic leukemia.

In some embodiments, the sample is a cell, a composition of cells, or a biological fluid. The sample may be obtained from a cell culture, a tissue, or an organism. In certain embodiments, sample is obtained from bone marrow, peripheral blood, or a tumor. Methods known to those of skill in the art may be used to obtain a sample from a subject. For example, a sample may be obtained by biopsy, aspiration, surgical resection, or venipuncture.

Expression may be determined by any method known to those of skill in the art. In certain aspects of the invention, expression is evaluated by assaying transcription levels. In other aspects of the invention, expression is evaluated by assaying protein levels.

TABLE 1
Proteins that are differentially expressed between
drug-resistant and drug-sensitive cells.

| Protein Name | Data Bank | Accession # |
|---|---|---|
| annexin A10 | NCBI | 20141284 |
| XEDAR | SWISS | Q9HAV5 |
| Hypothetical Protein | NCBI | 11360186M |
| (DKFZp564L1878.1) | | |
| (Tumoregulin) | | |
| P52rIPK protein homolog | NCBI | 7513233M |
| (DKFZp564B102.1) | | |
| ADP-ribosyl cyclase 1 | SWISS | P28907 |
| (CD38) | | |
| connective tissue growth | NCBI | 14626956 |
| factor (CTFG) | | |
| CD28 | SWISS | P10747 |
| BCL2-related ovarian killer | NCBI | 14210524M |
| Tumor necrosis factor | SWISS | Q9UBN6 |
| receptor superfamily | | |
| member 10D precursor | | |
| (DcR2) | | |
| CHROMOSOME 21 OPEN | NCBI | NP 478067 |
| READING FRAME 63 | | |
| (PRED34) | | |
| Hypothetical zinc finger | SWISS | Q8TF45 |
| protein KIAA 1956 | | |
| Hypothetical zinc finger | SWISS | O14628 |
| protein KIAA 195 | | |
| Zinc finger protein 42 | SWISS | P28698 |
| (Myeloid zinc finger 1) | | |
| (MZF-1) | | |
| Zinc finger protein 147 | SWISS | Q14258 |
| (Estrogen responsive finger | | |
| protein) | | |
| similar to Zinc finger | SWISS | Q99676 |
| protein 184 | | |
| Zinc finger protein-7 | SWISS | P17097 |
| (KOX4) | | |
| Krueppel-related zinc finger | SWISS | P10074 |
| protein 3 (HKLR3 protein) | | |
| zinc finger protein 336 | SWISS | Q9H116 |
| zinc finger protein 16 (KOX | SWISS | P17020 |
| 9) | | |
| zinc finger protein 74 | NCBI | 12643294M |
| Zinc finger 51 (BCL-6) | NCBI | 21040324M |
| Zinc finger protein 23 (Zinc | SWISS | P17027 |
| finger protein KOX16) | | |
| zinc finger protein 189 | SWISS | O75820 |
| zinc finger protein 179 | SWISS | Q9ULX5 |
| zinc finger protein 221 | SWISS | Q9UK13 |
| zinc finger protein 180 | SWISS | Q9UJW8 |
| (HHZ168) | | |
| Zinc finger protein 255 | SWISS | Q9UID9 |
| (Bone marrow zinc finger | | |
| 2) | | |
| Zinc finger protein 175 | SWISS | Q9Y473 |
| (Zinc finger protein | | |
| OTK18) | | |
| zinc finger protein 234 | SWISS | Q14588 |
| zinc finger protein 304 | SWISS | Q9HCX3 |
| Zinc finger protein 45 | SWISS | Q02386 |
| (BRC1744) | | |
| Zinc finger protein 234 | SWISS | Q14588 |
| (HZF4) | | |
| PR-domain zinc finger | SWISS | Q9H4Q3 |
| protein 13 | | |

In one embodiment, the present invention provides a method for identifying potential drug targets or drug templates in a cancer cell resistant to an anti-cancer agent comprising: obtaining a first cancer cell, wherein the first cancer cell is sensitive to the anti-cancer agent; obtaining a second cancer cell, wherein the second cancer cell is resistant to the anti-cancer agent; and identifying proteins that are differentially expressed between the first cancer cell and the second cancer cell to identify potential drug targets or drug templates.

In one embodiment, the present invention provides a method for identifying potential drug targets or drug templates in a cancer cell resistant to an Abl kinase inhibitor comprising: obtaining a first cancer cell, wherein the first cancer cell is sensitive to the Abl kinase inhibitor; obtaining a second cancer cell, wherein the second cancer cell is resistant to the Abl kinase inhibitor; and identifying proteins that are differentially expressed between the first cancer cell and the second cancer cell to identify potential drug targets or drug templates.

In certain aspects of the invention, the cancer cell is a Philadelphia-chromosome positive cell. In some embodiments, the cancer cell is a leukemia cell.

In one embodiment, the present invention provides a method of screening a candidate substance for anti-cancer activity comprising: contacting a first cell with the candidate substance; and evaluating the expression of one or more of the proteins in Table 2 or Table 3 in the first cell in the presence of the candidate substance to screen the candidate substance for anti-cancer activity. In some embodiments, the method further comprises comparing the expression of one or more of the proteins in Table 2 or Table 3 in the first cell in the presence of the candidate substance with the expression in a second cell in the absence of the candidate substance. The expression of the proteins in Table 2 and Table 3 may be evaluated at the protein level or at the mRNA level. An increase in the expression of one or more of the proteins in Table 2 and/or a decrease in the expression of one or more of the proteins in Table 3 in the presence of the candidate substance is an indicator of anti-cancer activity.

TABLE 2
Proteins Up-Regulated in Drug-Sensitive Cells
Compared to Drug-Resistant Cells.

| Protein Name | Data Bank | Accession # |
|---|---|---|
| annexin A10 | NCBI | 20141284 |
| XEDAR | SWISS | Q9HAV5 |
| Hypothetical Protein | NCBI | 11360186M |
| (DKEZp564L1878.1) | | |
| (Tumoregulin) | | |
| P52rIPK protein homolog | NCBI | 7513233M |
| (DKFZp564B102.1) | | |
| ADP-ribosyl cyclase 1 | SWISS | P28907 |
| (CD38) | | |
| connective tissue growth | NCBI | 14626956 |
| factor (CTFG) | | |
| CD28 | SWISS | P10747 |
| BCL2-related ovarian killer | NCBI | 14210524M |
| Hypothetical zinc finger | SWISS | Q8TF45 |
| protein KIAA 1956 | | |
| Hypothetical zinc finger | SWISS | O14628 |
| protein KIAA 195 | | |
| Zinc finger protein 42 | SWISS | P28698 |
| (Myeloid zinc finger 1) | | |
| (MZF-1) | | |
| Zinc finger protein 147 | SWISS | Q14258 |
| (Estrogen responsive finger | | |
| protein) | | |
| similar to Zinc finger | SWISS | Q99676 |
| protein 184 | | |
| Zinc finger protein-7 | SWISS | P17097 |
| (KOX4) | | |
| Krueppel-related zinc finger | SWISS | P10074 |
| protein 3 (HKR3 protein) | | |
| zinc finger protein 336 | SWISS | Q9H116 |
| zinc finger protein 16 (KOX | SWISS | P17020 |
| 9) | | |
| zinc finger protein 74 | NCBI | 12643294M |
| Zinc finger 51 (BCL-6) | NCBI | 21040324M |
| Zinc finger protein 23 (Zinc | SWISS | P17027 |
| finger protein KOX16) | | |
| zinc finger protein 189 | SWISS | O75820 |
| zinc finger protein 179 | SWISS | Q9ULX5 |
| zinc finger protein 221 | SWISS | Q9UK13 |
| zinc finger protein 180 | SWISS | Q9UJW8 |
| (HHZ168) | | |
| Zinc finger protein 255 | SWISS | Q9UID9 |
| (Bone marrow zinc finger | | |
| 2) | | |
| Zinc finger protein 175 | SWISS | Q9Y473 |
| (Zinc finger protein | | |
| OTK18) | | |
| zinc finger protein 234 | SWISS | Q14588 |
| zinc finger protein 304 | SWISS | Q9HCX3 |
| Zinc finger protein 45 | SWISS | Q02386 |
| (BRC1744) | | |
| Zinc finger protein 234 | SWISS | Q14588 |
| (HZF4) | | |
| PR-domain zinc finger | SWISS | Q9H4Q3 |
| protein 13 | | |

TABLE 3
Proteins Down-Regulated in Drug-Sensitive Cells
Compared to Drug-Resistant Cells.

| Protein Name | Data Bank | Accession # |
|---|---|---|
| Tumor necrosis factor | SWISS | Q9UBN6 |
| receptor superfamily | | |
| member 10D precursor | | |
| (DcR2) | | |
| CHROMOSOME 21 OPEN | NCBI | NP 478067 |
| READING FRAME 63 | | |
| (PRED34) | | |

In certain aspects of the invention, candidate compounds can be tested for anti-cancer activity in a tissue culture system using cell lines that are resistant to an Abl kinase inhibitor, such as imatinib mesylate. For example, a cell line that carries a mutation in the ABL gene that renders the cells resistant could be used. Culturing these cells with the candidate compound and studying the levels of killing within 48 hr can provide information on the therapeutic value of the compound. In addition, animal models (transgenic mice or SCID mice) can also be used for testing new compounds.

In one embodiment, the present invention provides a method for identifying a compound that inhibits P58IPK interaction with PKR comprising obtaining a compound that is a candidate inhibitor of the interaction between P58IPK and PKR, combining the compound with P58IPK and PKR, and assessing whether the compound inhibits interaction between P58IPK and PKR. It is contemplated that any compound may be assessed for the ability to inhibit the interaction between P58IPK and PKR. The compound can be natural or synthetic. It can be a protein or fragment thereof, small molecule, or a nucleic acid molecule.

Inhibitors of the interaction between P58IPK and PKR may act in a variety of ways. For example, an inhibitor may directly block the physical interaction between P58IPK and PKR, sequester P58IPK away from PKR, downregulate the transcription or translation of P58IPK, or upregulate proteins such as P52rIPK and Hsp40, which interact with P58IPK in an inactive complex.

The ability of a compound to inhibit P58IPK interaction with PKR may be assessed in a cell or in a cell-free system. When screening a compound for its ability to inhibit P58IPK interaction with PKR, it may be desirable to assess the interaction between P58IPK and PKR in the presence and in the absence of the candidate compound. Accordingly, a decrease in the interaction between P58IPK and PKR in the presence of the candidate compound as compared to the level of interaction in the absence of the candidate compound indicates that the candidate compound is an inhibitor of the interaction between P58IPK and PKR.

Those of skill in the art will be familiar with methods for assessing the interaction between P58IPK and PKR. For example, interaction between P58IPK and PKR could be assessed using an in vitro binding assay. In a non-limiting example of an in vitro binding assay, one protein, for example PKR, is immobilized to a solid support such as a microtiter plate, CNBR activated paper, CNBR activated Sepharose column, magnetic bead or any affinity capture media. The other protein, for example P58IPK is conjugated with biotin or horseradish peroxidase, or any other reporter system whereby binding to the immobilized PKR would result in capture of the P58IPK and the captured protein could be visualized and quantitated by activation of the reporter system. The resulting fluorescence, chemiluminescence or calorimetric response is then measured for the binding.

In certain aspects, the method for identifying a compound that inhibits P58IPK interaction with PKR, further comprises manufacturing a pharmaceutical composition comprising the compound.

In one embodiment, the present invention provides pharmaceutical composition manufactured by a method comprising: obtaining a compound that is a candidate inhibitor of the interaction between P58IPK and PKR, combining the compound with P58IPK and PKR, and assessing whether the compound inhibits interaction between P58IPK and PKR.

In one embodiment, the present invention provides a method for inhibiting growth of a cancer cell comprising: identifying a compound that inhibits interaction between P58IPK and PKR; and contacting the cancer cell with the compound. In certain aspects, the cancer cell is in a subject. In particular embodiments, the subject is a mammal. In a preferred embodiment, the mammal is a human. In certain embodiments, the method further comprising contacting the cancer cell with a second anti-cancer agent, such as chemotherapy, radiotherapy, immunotherapy, or gene therapy. In some embodiments, the second anti-cancer agent is IFN-γ or imatinib mesylate.

In one embodiment, the present invention provides a method for inhibiting the growth of a cancer cell comprising contacting the cancer cell with an expression construct comprising a polynucleotide encoding a polypeptide listed in Table 3. In a preferred embodiment, the present invention provides a method for inhibiting the growth of a cancer cell comprising contacting the cancer cell with an expression construct comprising a polynucleotide encoding P52rIPK (GenBank Accession Number NM004705, incorporated herein by reference) or P52rIPK homolog DKFZp564B102.1.

In certain aspects, the cancer cell is in a subject. In particular embodiments, the subject is a mammal. In a preferred embodiment, the mammal is a human. In certain embodiments, the method further comprises contacting the cancer cell with a second anti-cancer agent, such as chemotherapy, radiotherapy, immunotherapy, or gene therapy. In some embodiments, the second anti-cancer agent is IFN-γ or imatinib mesylate.

In another embodiment, the present invention provides a method of screening a candidate compound for anti-cancer activity comprising: contacting a first cell with the candidate compound; and evaluating the expression of one or more of P52rIPK or P52rIPK homolog DKFZp564B102.1 in the first cell in the presence of the candidate compound to screen the candidate compound for anti-cancer activity. In some embodiments, the method further comprises comparing the expression of one or more of P52rIPK or P52rIPK homolog DKFZp564B102.1 in the first cell in the presence of the candidate compound with the expression of one or more of P52rIPK or P52rIPK homolog DKFZp564B102.1 in a second cell in the absence of the candidate compound. In certain embodiments, the method further comprises manufacturing a pharmaceutical composition comprising the candidate compound.

In one embodiment, the present invention provides a pharmaceutical composition comprising a compound identified by a method comprising: contacting a first cell with the candidate compound; and evaluating the expression of one or more of P52rIPK or P52rEPK homolog DKFZp564B102.1 in the first cell in the presence of the candidate compound to identify a candidate compound with anti-cancer activity. In certain embodiments, the method further comprises comparing the expression of one or more of P52rIPK or P52rIPK homolog DKFZp564B102.1 in the first cell in the presence of the candidate compound with the expression of one or more of P52rIPK or P52rIPK homolog DKFZp564B102.1 in a second cell in the absence of the candidate compound.

It is contemplated that any method or composition described herein can be implemented with respect to any other method or composition described herein.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: FIG. 1 illustrates the interaction of P52rIPK, P58IPK, PKR, and PERK in signal transduction. As can be seen in the figure, INF-γ initiates a signaling cascade involving PKR and eIF-2α, which results in the inhibition of translation initiation and induction of apoptosis. P58IPK is an inhibitor of PKR and PERK. Thus, P58IPK can block growth inhibition and apoptosis mediated by PKR and PERK. P52rIPK binds P58IPK, reversing inhibition of PKR and PERK mediated growth inhibition and apoptosis.

FIGS. 2A, 2B, 2C, and 2D: Proteins from bone marrow aspirates of patients with CML were separated by two-dimensional gel electrophoresis. FIG. 2A is a gel image showing the up and down regulated spots in the pH 4-7 range in a Gleevec-sensitive sample as compared to the gel image in FIG. 2B, which is from a Gleevec-resistant sample. A quantitative comparison of the average density in parts-per-million (PPM) of the up and down regulated spots in the pH 4-7 range from Gleevec-resistant samples versus Gleevec-sensitive is shown if FIG. 2C. FIG. 2D shows the approximate molecular weight (MW) and pI of the 7 spots that were consistently up or down regulated in Gleevec-sensitive samples versus Gleevec-resistant samples.

FIGS. 3A, 3B, 3C, and 3D: Proteins from bone marrow aspirates of patients with CML were separated by two-dimensional gel electrophoresis. FIG. 3A is a gel image showing 12 spots in the pH 6-11 range that were consistently up regulated in Gleevec-sensitive samples as compared to the gel image in FIG. 3B, which is from a Gleevec-resistant sample. FIG. 3C and FIG. 3D show a quantitative comparison of the average density in parts-per-million (PPM) of the up regulated spots in the pH 6-11 range from Gleevec-sensitive samples versus Gleevec-resistant samples.

### DESCRIPTION OF ILLUSTRATIVE EMBODIMENTS

A. The Present Invention

Drug resistance is a major obstacle in the treatment of cancer. Clinical experience shows that some cancers demonstrate selective sensitivity to certain drugs but resistance to others. Treatment decisions, however, are typically made empirically using a trial-and-error approach. The ability to select the optimal anti-cancer therapy from several alternative treatment options would be an important clinical advance. In addition, there is a need for new methods and therapeutic compositions that can overcome drug resistance or enhance the effectiveness of other anti-cancer agents.

The present invention overcomes the deficiencies in the prior art by providing methods and compositions for identifying cancer cells that are either sensitive or resistant to a particular anti-cancer therapy. Accordingly, the present invention allows for more accurate diagnosis, prognosis, and monitoring of a subject's condition. Furthermore, the ability to assess a subject's resistance or sensitivity to a particular treatment regimen will permit more informed treatment decisions to be made prior to beginning therapy. The present invention also overcomes deficiencies in the prior art concerning the treatment of cancers by providing methods and compositions for treating cancer and improving the effectiveness of other cancer therapies.

B. Hyperproliferative Diseases

The present invention may be used in the diagnosis, prognosis, monitoring, and treatment of hyperproliferative diseases including, but not limited to, cancer. A hyperproliferative disease is any disease or condition which has, as part of its pathology, an abnormal increase in cell number. Included in such diseases are benign conditions such as benign prostatic hypertrophy and ovarian cysts. Also included are premalignant lesions, such as squamous hyperplasia. At the other end of the spectrum of hyperproliferative diseases are cancers. A hyperproliferative disease can involve cells of any cell type. The hyperproliferative disease may or may not be associated with an increase in size of individual cells compared to normal cells.

Another type of hyperproliferative disease is a hyperproliferative lesion, a lesion characterized by an abnormal increase in the number of cells. This increase in the number of cells may or may not be associated with an increase in size of the lesion. Examples of hyperproliferative lesions that are contemplated for treatment include benign tumors and premalignant lesions. Examples include, but are not limited to, squamous cell hyperplastic lesions, premalignant epithelial lesions, psoriatic lesions, cutaneous warts, periungual warts, anogenital warts, epidermdysplasia verruciformis, intraepithelial neoplastic lesions, focal epithelial hyperplasia, conjunctival papilloma, conjunctival carcinoma, or squamous carcinoma lesion. The lesion can involve cells of any cell type. Examples include keratinocytes, epithelial cells, skin cells, and mucosal cells.

The term “cancer” as used herein is defined as a tissue of uncontrolled growth or proliferation of cells, such as a tumor. Cancer develops through the accumulation of genetic alterations (Fearon and Vogelstein, 1990) and gains a growth advantage over normal surrounding cells. The genetic transformation of normal cells to neoplastic cells occurs through a series of progressive steps. Genetic progression models have been studied in some cancers, such as head and neck cancer (Califano et al., 1996).

Examples of cancers include, but are not limited to, breast cancer, lung cancer, prostate cancer, ovarian cancer, brain cancer, liver cancer, cervical cancer, colon cancer, renal cancer, skin cancer, head & neck cancer, bone cancer, esophageal cancer, bladder cancer, uterine cancer, lymphatic cancer, stomach cancer, pancreatic cancer, testicular cancer, or leukemia.

Leukemia is of particular interest in the context of the present invention. Leukemia is a hematologic malignancy characterized by abnormal proliferation of leukocytes. The disease is classified according to the type of leukocyte most prominently involved. Acute leukemias are predominantly undifferentiated cell populations and chronic leukemias have more mature cell forms. The acute leukemias are divided into lymphoblastic (ALL) and non-lymphoblastic (ANLL) types and may be further subdivided by morphologic and cytochemical appearance according to the French-American-British classification or according to their type and degree of differentiation. Specific B- and T-cell, as well as myeloid cell surface markers/antigens are used in the classification too. ALL is predominantly a childhood disease while ANLL, also known as acute myeloid leukemia (AML), is a more common acute leukemia among adults.

Chronic leukemias are divided into lymphocytic (CLL) and myeloid (CML) types. CLL is characterized by the increased number of mature lymphocytes in blood, bone marrow, and lymphoid organs. Most CLL patients have clonal expansion of lymphocytes with B cell characteristics. CLL is a disease of older persons. In CML, the granulocytic cells predominate at all stages of differentiation in blood and bone marrow, but may also affect liver, spleen, and other organs.

Although a number of anti-cancer agents are available for the treatment of cancer, cancer cell resistance to these agents remains a major problem in clinical oncology. It is unclear why a cancer cell may be resistant to an anti-cancer agent. The ability to predict, prevent, or delay resistance would be a valuable tool for the treatment of cancer.

C. ABL Kinase Inhibitors

The present invention provides methods and compositions useful for identifying proteins or protein patterns associated with sensitivity or resistance to an Abl kinase inhibitor. The ability to accurately predict whether a patient will be sensitive or resistant to a particular therapy will result in treatment strategies that are better tailored to the individual's needs. In addition, the present invention also provides proteins and protein patterns known to associate with Abl kinase inhibitor resistance and sensitivity.

A reciprocal translocation, between human chromosomes 9 and 22, known as Philadelphia chromosome, results in an abnormal BCR-ABL fusion gene. BCR-ABL-mediated tyrosine phosphorylation promotes transformation of hematopoeietic progenitor cells into chronic myeloid and acute lymphocytic leukemias. Knowledge of the function of the BCR-ABL fusion gene led to the development of the small molecule drug, imatinib mesylate (Gleevec®). Imatinib mesylate has proved successful in the treatment of patients with CML (Druker et al., 1996; Druker et al., 2000). Imatinib mesylate binds to the BCR-ABL protein and inhibits its kinase activity, thus controlling diseases driven by this kinase. Despite the dramatic success achieved in treating CML with imatinib mesylate, patients frequently relapse and progress on therapy (Sawyers et al., 2002; Talpaz et al., 2002; Druker et al., 2001).

Imatinib mesylate is also a potent inhibitor of three other tyrosine kinases, namely KIT, platelet-derived growth factor receptors (PDGFR-A and B), and the Abelson-related gene (ARG). Accordingly, diseases associated with activated KIT, PDGFR-A and B, or ARG may be amenable to treatment with imatinib mesylate. For example, Gleevec® has received FDA approval for use in the treatment of patients with KIT-positive gastrointestinal stromal tumors (GIST).

Criteria for evaluating response to imatinib mesylate may be defined at the hematologic or cytogenetic level. A patient may be regarded as having a hematologic response if he has achieved normal leukocyte and platelet levels within three months of starting imatinib mesylate treatment. A patient may be regarded as having a cytogenetic response if within twelve months of starting imatinib mesylate treatment no Philadelphia chromosome positive cells are observed on examination of 20 bone marrow metaphases. A patient may be regarded as a potential responder if after three months of imatinib mesylate treatment he has achieved a cytogenetic response of greater than 35% Philadelphia chromosome negative metaphases, and thereafter he continues to achieve a further 30% or more reduction in the percentage of Philadelphia chromosome positive metaphases at each three month interval.

D. Protein Analysis

The present invention employs methods of separating proteins. Methods of separating proteins are well known to those of skill in the art and include, but are not limited to, various kinds of chromatography (e.g., anion exchange chromatography, affinity chromatography, sequential extraction, and high performance liquid chromatography), and mass spectrometry.

1. Two-Dimensional Electrophoresis

In one embodiment the present invention employs two-dimensional gel electrophoresis to separate proteins from a biological sample into a two-dimensional array of protein spots.

Two-dimensional electrophoresis is a useful technique for separating complex mixtures of molecules, often providing a much higher resolving power than that obtainable in one-dimension separations. Two-dimensional gel electrophoresis can be performed using methods known in the art (See, e.g., U.S. Pat. Nos. 5,534,121 and 6,398,933). Typically, proteins in a sample are separated first by isoelectric focusing, during which proteins in a sample are separated in a pH gradient until they reach a spot where their net charge is zero (i.e., isoelectric point). This first separation step results in a one-dimensional array of proteins. The proteins in the one-dimensional array are further separated using a technique generally distinct from that used in the first separation step. For example, in the second dimension proteins may be further separated by polyacrylamide gel electrophoresis in the presence of sodium dodecyl sulfate (SDS-PAGE). SDS-PAGE allows further separation based on the molecular mass of the protein.

Proteins in the two-dimensional array can be detected using any suitable methods known in the art. Staining of proteins can be accomplished with calorimetric dyes (e.g., coomassie), silver staining, or fluorescent staining (Ruby Red; SyproRuby). As is known to one of ordinary skill in the art, spots or protein patterns generated can be further analyzed. For example, proteins can be excised from the gel and analyzed by mass spectrometry. Alternatively, the proteins can be transferred to an inert membrane by applying an electric field and the spot on the membrane that approximately corresponds to the molecular weight of a marker can be analyzed by mass spectrometry.

2. Mass Spectrometry

In certain embodiments the present invention employs mass spectrometry. Mass spectrometry provides a means of “weighing” individual molecules by ionizing the molecules in vacuo and making them “fly” by volatilization. Under the influence of combinations of electric and magnetic fields, the ions follow trajectories depending on their individual mass (m) and charge (z). The “time of flight” of the ion before detection by an electrode is a measure of the mass-to-charge ratio (m/z) of the ion. Mass spectrometry (MS), because of its extreme selectivity and sensitivity, has become a powerful tool for the quantification of a broad range of bioanalytes including pharmaceuticals, metabolites, peptides and proteins.

Matrix-assisted laser desorption ionization-time of flight mass spectrometry (MALDI-TOF MS) is a type of mass spectrometry in which the analyte substance is distributed in a matrix before laser desorption. MALDI-TOF MS has become a widespread analytical tool for peptides, proteins and most other biomolecules (oligonucleotides, carbohydrates, natural products, and lipids). In combination with 2D-gel electrophoresis, MALDI-TOF MS is particularly suitable for the identification of protein spots by peptide mass fingerprinting or microsequencing.

In MALDI-TOF analysis, the analyte is first co-crystallized with a matrix compound, after which pulse UV laser radiation of this analyte-matrix mixture results in the vaporization of the matrix which carries the analyte with it. The matrix therefore plays a key role by strongly absorbing the laser light energy and causing, indirectly, the analyte to vaporize. The matrix also serves as a proton donor and receptor, acting to ionize the analyte in both positive and negative ionization modes. A protein can often be unambiguously identified by MALDI-TOF analysis of its constituent peptides (produced by either chemical or enzymatic treatment of the sample).

Another type of mass spectrometry is surface-enhanced laser desorption ionization-time of flight mass spectrometry (SELDI-TOF MS). Whole proteins can be analyzed by SELDI-TOF MS, which is a variant of MALDI-TOF MS. In SELDI-TOF MS, fractionation based on protein affinity properties is used to reduce sample complexity. For example, hydrophobic, hydrophilic, anion exchange, cation exchange, and immobilized-metal affinity surfaces can be used to fractionate a sample. The proteins that selectively bind to a surface are then irradiated with a laser. The laser desorbs the adherent proteins, causing them to be launched as ions. The SELDI-TOF MS approach to protein analysis has been implemented commercially (e.g., Ciphergen).

Tandem mass spectrometry (MS/MS) is another type of mass spectrometry known in the art. With MS/MS analysis ions separated according to their m/z value in the first stage analyzer are selected for fragmentation and the fragments are then analyzed in a second analyzer. Those of skill in the art will be familiar with protein analysis using MS/MS, including QTOF, Ion Trap, and FTMS/MS. MS/MS can also be used in conjunction with liquid chromatography via electrospray or nanospray interface or a MALDI interface, such as LCMS/MS, LCLCMS/MS, or CEMS/MS.

3. Other Methods of Protein Analysis

In addition to the methods described above, other methods of protein separation and analysis known in the art may be used in the practice of the present invention. The methods of protein of protein separation and analysis may be used alone or in combination.

Of particular interest are various forms of chromatography. Chromatography is used to separate organic compounds on the basis of their charge, size, shape, and solubilities. Chromatography consists of a mobile phase (solvent and the molecules to be separated) and a stationary phase either of paper (in paper chromatography) or glass beads, called resin, (in column chromatography) through which the mobile phase travels. Molecules travel through the stationary phase at different rates because of their chemistry. Types of chromatography that may be employed in the present invention include, but are not limited to, high performance liquid chromatography (HPLC), ion exchange chromatography (IEC), and reverse phase chromatography (RP). Other kinds of chromatography that may be used include: adsorption, partition, affinity, gel filtration, and molecular sieve, and many specialized techniques for using them including column, paper, thin-layer, and gas chromatography (Freifelder, 1982).

4. Analysis of Protein Markers and Patterns

Following separation of the proteins, the protein markers and protein patterns may be further analyzed. Where, for example, the protein markers have been separated by two-dimensional gel electrophoresis, the protein markers may be visualized by staining the gel. Protein standards having known molecular weights and isoelectric focusing points can be used as landmarks. Gels are preferably stained by Spyro Ruby fluorescent dye. Other dyes, such as silver staining and coomassie blue, are known in the art and could be used.

Gel images may be compared visually and/or electronically. To compare gel images electronically, the gels are first scanned (e.g., Molecular Imager FX (Bio-Rad Laboratories)) and then analyzed using software such as PDQUEST (Bio-Rad Laboratories). Analysis includes spot normalization, spot detection, and comparisons of protein patterns. Spot density may be quantitatively normalized based on the density of each spot versus the total density of all detected spots. The image analysis software may be set up for the analysis of PPM for each spot and also for highlighting fold differences between spots in any set of image comparisons.

In one aspect of the invention, the gel images are compared between a drug-resistant cell and a drug-sensitive cell to identify protein markers and protein patterns that differ between the two. In other aspects of the invention, the gel images are compared between a first cell of unknown drug sensitivity or resistance and a reference cell of known drug sensitivity or resistance to characterize the first cell as either drug-sensitive or drug resistant.

Following differential expression analysis, spots of interest can be excised from the gel for identification. Those of skill in the art will be familiar with methods, such as mass fingerprinting analysis and microsequencing, which may be used to identify the protein spots. In a preferred embodiment, the ProteomeWorks robotic spot cutter (Bio-Rad Laboratories) is used to excise the spots from the gel. Excised spots are then in-gel digested on a MultiPROBE II (Packard, Downers Grove, Ill.). The gel is then re-hydrated and the digested peptides are extracted from the gel.

Mass spectral analyses of the digested peptides can be performed to identify the protein markers. Those of skill in the art are familiar with mass spectral analysis of digested peptides. In a preferred embodiment, mass spectral analysis is conducted on MALDI-TOF Voyager DE PRO (Applied Biosystems). Spectra should be carefully scrutinized for acceptable signal-to-noise ratio (S/N) to eliminate spurious artifact peaks from the peptide molecular weight lists. Both internal and external standards may be employed. The internal or external standards are considered for calibration of any shift in mass values during mass spectroscopic analysis. External standards are a set proteins of known molecular weight and known m/z value in the mass spectrum. A mixture of external standards is placed on the mass spec chip well next to the well that includes a desired sample. Internal standards are characteristic peaks in the sample spectrum that belong to peptides of the proteolytic enzyme (e.g., trypsin) used to digest protein spots and extracted along with the digested peptides. Those peaks are used for internal calibration of any deviation of spectral peaks of the sample.

Corrected molecular weight lists can then be subjected to database searches (e.g., NCBI and Swiss Protein data banks). Those of skill in the art are familiar with searching databases like NCBI and Swiss Protein. In a preferred embodiment, values are set with a minimum matching peptide setting of 4, mass tolerance settings of 50-250 ppm, and for a single trypsin miss-cut.

E. Mechanism of Drug Resistance

The inventors discovered that homologs of P52rIPK are down-regulated in Gleevec-resistant cells from CML patients. This observation led the inventors to a mechanism for drug resistance and drug sensitivity from which novel methods and compositions for the treatment of leukemia and other cancers can be developed.

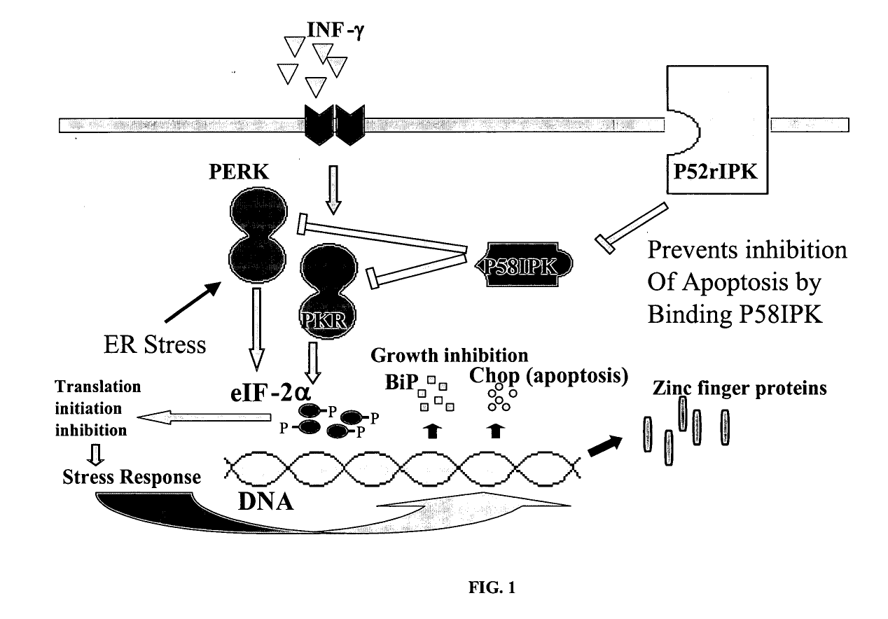

P52rIPK is a 52 kDa protein that acts as a growth suppressor and apoptotic activator via up regulation of PKR and PERK mediated eIF-2α phosphorylation. P52rIPK accomplishes this by interacting with P58IPK (GenBank Accession Number NM006260, incorporated herein by reference) overcoming that protein's interaction with and inhibition of PKR and PERK mediated growth suppression and apoptosis (Gale et al., 1998, incorporated by reference). FIG. 1 illustrates the interaction of these proteins in the INF-γ signal transduction pathway.

P52rIPK contains a 114 amino acid charged domain that exhibits homology to the charged domain of Hsp90 (Gale et al., 1998; Gale et al., 2002, incorporated by reference). The charged domain is necessary and sufficient for interaction with P58IPK. The charged domain of P52rIPK binds specifically to domain 7 of the P58IPK tetratricopeptide repeat (TPR), the domain adjacent to the TPR motif required for P58IPK interaction with PKR (Gale et al., 2002).

P52rIPK and its homologs provide novel drug templates for the development of drugs that can target P58IPK. These drugs would act to suppress cell growth and/or induce apoptosis, and therefore they would be useful in the treatment of conditions characterized by unregulated cell growth.

P58IPK is an Hsp40 family member known to inhibit protein kinase R (PKR) and PKR-like endoplasmic reticulum kinase (PERK) (Yan et al., 2002). P58IPK binds to and inactivates the kinase domain of both PKR and PERK. Overexpression of P58IPK has been shown to cause a transformed phenotype and rapid tumor formation in nude mice (Barber et al., 1994).

PKR is an IFN-induced, double-stranded RNA-activated kinase that mediates the antiviral and antiproliferative actions of IFN, in part via its translational inhibitory properties. Activation of PKR phosphorylates the α subunit of eukaryotic initiation factor-2 (eIF-2α), leading to a series of biochemical events that culminate in a dramatic decrease in the initiation of protein synthesis. In addition to its role in IFN-induced antiviral resistance, there is also evidence that PKR has tumor suppressor properties (see e.g., Barber et al., 1995; Koromilas et al., 1992); Meurs et al., 1993). PKR amino acids 244 to 296 contain the binding site for a select group of specific inhibitors including the cellular protein P58IPK (Tan et al., 1998).

PERK is an eIF-2α kinase that is activated in response to the accumulation of unfolded proteins in the endoplasmic reticulum (ER). PERK-mediated phosphorylation of eIF-2α attenuates protein synthesis to reduce ER client-protein load while selectively promoting the expression of certain genes such as BiP and Chop.

P58IPK provides an attractive target for the treatment of drug resistant cancers and for the enhancement of the effectiveness of anti-cancer agents in general. Accordingly, the present invention provides methods and compositions for identifying compounds that inhibit P58IPK. Of particular interest, are compounds that inhibit the interaction between P58IPK and PKR. Inhibiting this interaction will promote growth inhibition and apoptosis in the cell. Because compounds identified by the methods of the present invention promote growth inhibition and apoptosis, it is contemplated that they would be useful in the treatment of any type of cancer or other hyerproliferative disease.

F. Rational Drug Design

As mentioned above, P52rIPK and its homologs provide novel drug templates for the development of drugs that can target P58IPK. In addition, P58IPK provides an attractive target for the treatment of drug resistant cancers and for the enhancement of the effectiveness of anti-cancer agents in general. Accordingly, the present invention provides methods for screening candidate compounds for the ability to inhibit P58IPK.

One approach that can be used is rational drug design. Rational drug design involves making predictions relating to the structure of the target molecules and the candidate compound. In addition, rational drug design involves creating and examining the action of such compounds. The candidate compound may be a protein or fragment thereof, a small molecule, or even a nucleic acid molecule. It may prove to be the case that the most useful pharmacological compounds will be compounds that are structurally related to compounds which interact naturally with P58IPK, P52rIPK, or PKR. Thus, for example, compounds that are structurally similar to all or part of P52rIPK or a P52rIPK homolog such as DKFZp564B102.1 may be pharmaceutically useful compounds for inhibiting the interaction of P58IPK with PKR. Likewise, compounds that are structurally similar to all or part of PKR may also be useful for inhibiting the interaction of P58IPK with PKR.

The goal of rational drug design is to produce structural analogs of biologically active polypeptides or target compounds. By creating such analogs, it is possible to fashion drugs which are more active or stable than the natural molecules, which have different susceptibility to alteration or which may affect the function of various other molecules. This could be accomplished by x-ray crystallography, computer modeling or by a combination of both approaches.

It also is possible to use antibodies to ascertain the structure of a target compound. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically or biologically produced peptides. Selected peptides would then serve as the pharmacore.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled on active, but otherwise undesirable compounds.

Candidate compounds may include fragments or parts of naturally occurring compounds or may be found as active combinations of known compounds that are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds.

Yet further, the candidate substance may be a nucleic acid ligand, also referred to as an aptamer. Aptamers are short, single-stranded oligonucleotides that assume stable conformations and bind tightly to specific targets, including proteins. Thus, for example, an aptamer specific to P58IPK could be used to bind that protein and block it from physically interacting with PKR.

It will, of course, be understood that all the screening methods of the present invention are useful in themselves notwithstanding the fact that effective candidates may not be found. The invention provides methods for screening for such candidates, not solely methods of finding them.

G. Nucleic Acid-Based Expression Systems

The present invention provides methods for inhibiting the growth of a cancer cell comprising contacting the cancer cell with an expression construct comprising a polynucleotide encoding a polypeptide listed in Table 3. In a preferred embodiment, the expression construct comprises a polynucleotide encoding P52rIPK or a P52rIPK homolog such as DKFZp564B102.1. Those of skill in the art are familiar with methods of making expression constructs and administering them to a cell.

1. Vectors

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1990 and Ausubel et al., 1996, both incorporated herein by reference).

The term “expression vector” refers to any type of genetic construct comprising a nucleic acid coding for an RNA capable of being transcribed and then translated into a protein, polypeptide, or peptide. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra.

a. Promoters and Enhancers

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment.

A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are commonly used in recombinant DNA construction include the β-lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, it is contemplated the control sequences that direct transcription and/or expression of sequences within non-nuclear organelles, such as mitochondria, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 2001, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally any promoter/enhancer combination (as per, for example, the Eukaryotic Promoter Data Base EPDB, http://www.epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

The identity of tissue-specific promoters or elements, as well as assays to characterize their activity, is well known to those of skill in the art. Non-limiting examples of such regions include the human LIMK2 gene (Nomoto et al. 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al., 1998), D1A dopamine receptor gene (Lee, et al., 1997), insulin-like growth factor II (Wu et al., 1997), and human platelet endothelial cell adhesion molecule-1 (Almendro et al., 1996).

b. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

The use of internal ribosome entry sites (IRES) elements may be used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

c. Multiple Cloning Sites

Vectors can include a multiple cloning site (MCS), which is a nucleic acid region that contains multiple restriction enzyme sites, any of which can be used in conjunction with standard recombinant technology to digest the vector (see, for example, Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

d. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see, for example, Chandler et al., 1997, herein incorporated by reference.)

e. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to be more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

f. Polyadenylation Signals

In expression, particularly eukaryotic expression, one will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal or the bovine growth hormone polyadenylation signal, convenient and known to function well in various target cells. Polyadenylation may increase the stability of the transcript or may facilitate cytoplasmic transport.

g. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

h. Selectable and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

H. Pharmaceutical Preparations

1. Formulations

Pharmaceutical preparations of the compounds and expression constructs of the present invention are also contemplated. One of ordinary skill in the art would be familiar with techniques for administering pharmaceutical preparations to a subject. Furthermore, one of ordinary skill in the art would be familiar with techniques and pharmaceutical reagents necessary for preparation of these compounds prior to administration to a subject.

Aqueous compositions of the present invention comprise an effective amount of a compound or expression construct in a pharmaceutically acceptable carrier or aqueous medium. As used herein, “pharmaceutical preparation” or “pharmaceutical composition” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the compound or expression construct, its use in the therapeutic compositions is contemplated. Supplementary active ingredients can also be incorporated into the compositions. For human administration, preparations should meet sterility, pyrogenicity, general safety and purity standards as required by the FDA Center for Biologics.

The compounds and expression constructs of the present invention may be formulated for administration by any known route, such as parenteral administration. Determination of the amount of a compound or expression construct to be administered will be made by one of skill in the art, and will in part be dependent on the extent and severity of cancer. The preparation of the pharmaceutical composition containing a compound or an expression construct of the invention disclosed herein will be known to those of skill in the art in light of the present disclosure.

The present invention contemplates compounds and expression constructs that will be in pharmaceutical preparations that are sterile solutions for subcutaneous injection, intramuscular injection, intravascular injection, intratumoral injection, or application by any other route. A person of ordinary skill in the art would be familiar with techniques for generating sterile solutions for injection or application by any other route.

Upon formulation, solutions will be administered in a manner compatible with the dosage formulation and in such amount as is therapeutically effective. The formulations are easily administered in a variety of dosage forms, such as the type of injectable solutions described above. For parenteral administration, the pharmaceutical composition should be suitably buffered. The compounds and expression constructs of the present invention may be administered with other agents that are part of the therapeutic regiment of the subject, such as radiotherapy, immunotherapy, gene therapy, or chemotherapy.

2. Dosage

The present invention contemplates administration of a therapeutic composition a subject for the treatment of cancer and other hyperproliferative diseases. One of ordinary skill in the art would be able to determine the amount to be administered and the frequency of administration in view of this disclosure. The quantity to be administered, both according to number of treatments and dose, also depends on the judgment of the practitioner and are peculiar to each individual.

In certain embodiments, it may be desirable to provide a continuous supply of the therapeutic compositions to the patient. Continuous perfusion of the region of interest (such as the tumor) may be preferred. The time period for perfusion would be selected by the clinician for the particular patient and situation, but times could range from about 1-2 hours, to 2-6 hours, to about 6-10 hours, to about 10-24 hours, to about 1-2 days, to about 1-2 weeks or longer. Generally, the dose of the therapeutic composition via continuous perfusion will be equivalent to that given by single or multiple injections, adjusted for the period of time over which the doses are administered.

I. Combination Treatments

In order to increase the effectiveness of the compounds and expression constructs of the present invention as a cancer therapy, it may be desirable to combine treatment with other agents effective in the treatment of cancer. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This process may involve contacting the cells with the compound or expression construct and another agent(s) at the same time. This may be achieved by contacting the cell with a single composition or pharmacological formulation that includes both agents, or by contacting the cell with two distinct compositions or formulations, at the same time, wherein one composition includes a compound or expression construct of the present invention and the other includes the second agent(s).

Tumor cell resistance to chemotherapy agents represents a major problem in clinical oncology. One goal of current cancer research is to find ways to improve the efficacy of chemotherapy. In the context of the present invention, it is contemplated that the compounds and expression constructs of the present invention could be used in conjunction with chemotherapeutic intervention. It is also contemplated that the compounds and expression constructs of the present invention could be used in conjunction with other forms of intervention.

1. Chemotherapy

Cancer therapies include a variety of combination therapies with both chemical and radiation based treatments. One of ordinary skill in the art would be familiar with the range of chemotherapeutic agents and combinations that are available. Chemotherapeutic agents include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, or any analog or derivative variant of the foregoing.

2. Radiotherapy

Other factors that cause DNA damage and have been used extensively include y-rays, X-rays, and the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

3. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

4. Genes

The secondary treatment may be a gene therapy. For example, the gene therapy can be a vector encoding a tumor suppressor. Examples of tumor suppressor include, p53, Rb, p16, MDA-7, PTEN and C-CAM.

5. Surgery

Many patients with cancer undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to the physical removal of at least part of a tumor. In addition to tumor resection, treatment by surgery includes laser surgery, cryosurgery, electrosurgery, and micrographic surgery (Mohs' surgery).

Upon excision of part or all of the cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, or 5 weeks or every 1, 2, 3, 4, 5, 6, 7, 8, 9, 10, 11, or 12 months. These treatments may be of varying dosages as well.

6. Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers.

It is further contemplated that the upregulation of cell surface receptors or their ligands such as Fas/Fas ligand, DR4 or DR5/TRAIL would potentiate the apoptotic inducing abilities of the present invention by establishment of an autocrine or paracrine effect on hyperproliferative cells. Increases in intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyerproliferative efficacy of the treatments. Inhibitors of cell adhesion, such as integrin and cadherin blocking antibodies, are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

### J. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Differentially Expressed Proteins in Gleevec-Sensitive and Gleevec-Resistant CML Resolved by 2-D Gel Electrophoresis

Bone marrow aspirate samples from 8 Gleevec sensitive and 6 Gleevec resistant CML patients were analyzed for differential protein expression using 2-D gel electrophoresis.

Two-Dimensional Gel Electrophoresis. Bone marrow aspirate samples were subjected to ProteEx protocol for protein purification and quantitative assay (U.S. patent application Ser. No. 10/301,512, incorporated by reference). Protein separation was conducted as mentioned before (Kuncewicz et al., 2003). Briefly, the purified proteins were suspended in a buffer containing 8 M urea, 2 M thiourea, 1% Triton X-100, 1% DTT, and 1% ampholytes pH 3-10. An aliquot of 100 μg of protein was loaded onto an 11 cm IEF strip (Bio-Rad Laboratories, Hercules Calif.), pH 4-7 and 6-11. Focusing was conducted on IEF cells (Bio-Rad Laboratories) at 250 V for 20 minutes followed by a linear increase to 8000 V for 2 hours. The focusing was terminated at 20,000 volt-hours.

Strips were then equilibrated in 375 mM Tris buffer, pH 8.8, containing 6 M urea, 20% glycerol, and 2% SDS. Fresh DTT was added to the buffer at a concentration of 30 mg/ml and incubated for 15 minutes, followed by an additional 15-minute incubation with fresh buffer containing 40 mg/ml iodoacetamide. Strips were then loaded onto the second dimension using Criterion pre-cast gradient gels (Bio-Rad Laboratories) with an acrylamide gradient of 10-20%. Gels were then stained using SyproRuby fluorescent dye.

Gel Image Analysis. Stained gels were scanned on laser scan Molecular imager FX (Bio-Rad Laboratories). The results of digital fluorescent image analysis of gel images from Gleevec-sensitive samples were compared to Gleevec-resistant samples by qualitative and quantitative comparison of protein patterns using pre-mixed internal protein standards (BioRad Laboratories) as landmarks.

Spot density was quantitatively normalized based on the density of each spot versus the total density of all detected spots. The software was set up for analysis of PPM for each spot and also for highlighting fold differences between spots in any set of image comparison. A reproducible density difference was considered significant with a coefficient of variation of <20%.

Tryptic Digestion, MALDI-TOF MS, and Peptide Mass Fingerprinting Analysis. Following differential expression analysis of the proteins, spots of interest were excised from the gel using the ProteomeWorks robotic spot cutter (Bio-Rad Laboratories). Excised spots were robotically in-gel digested on a MultiPROBE II (Packard, Downers Grove, Ill.) as follows: gel spots were washed twice in 100 mM NH₄HCO₃ buffer, followed by soaking in 100% acetonitrile for 5 minutes, aspiration of the acetonitrile, and drying of the gels for 30 minutes.

Re-hydration of the gels using 20 μg/ml trypsin (Promega, Madison, Wis.) suspended in 25 mM NH₄HCO₃ buffer was followed by incubation at 37° C. for 14-20 hours. The digested peptides were extracted twice using a solution of 50% acetonitrile and 5% trifluoroacetic acid for 40 minutes. Peptide extracts were desalted and concentrated using reverse phase C18 Zip-tips (Millipore, Bedford, Mass.) and robotically placed on MALDI chips using the SymBiot I (Applied Biosystems, Foster City, Calif.).

Mass spectral analyses were conducted on MALDI-TOF Voyager DE PRO (Applied Biosystems). Spectra were carefully scrutinized for acceptable signal-to-noise ratio (S/N) to eliminate spurious artifact peaks from the peptide molecular weight lists and both internal and external standards were employed. Corrected lists were subjected to database searches using both the NCBI and Swiss protein data banks with a minimum matching peptide setting of 4, mass tolerance settings of 50-250 ppm, and for a single trypsin miss-cut.

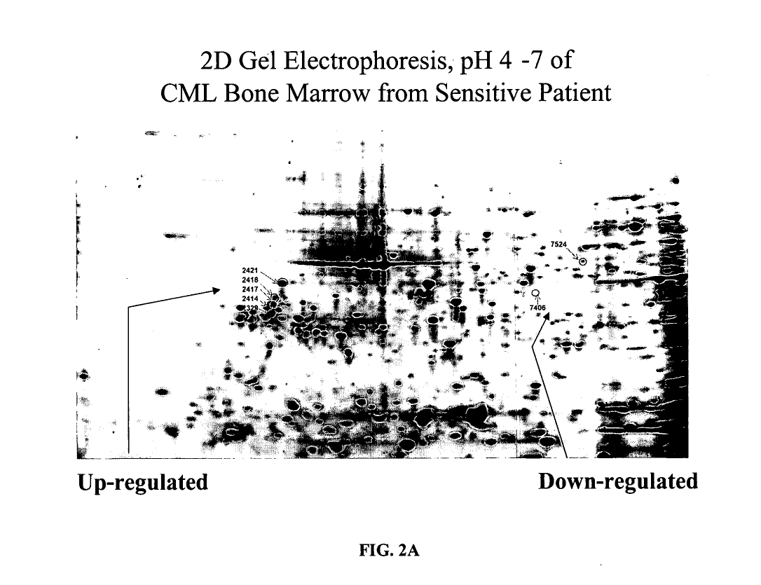

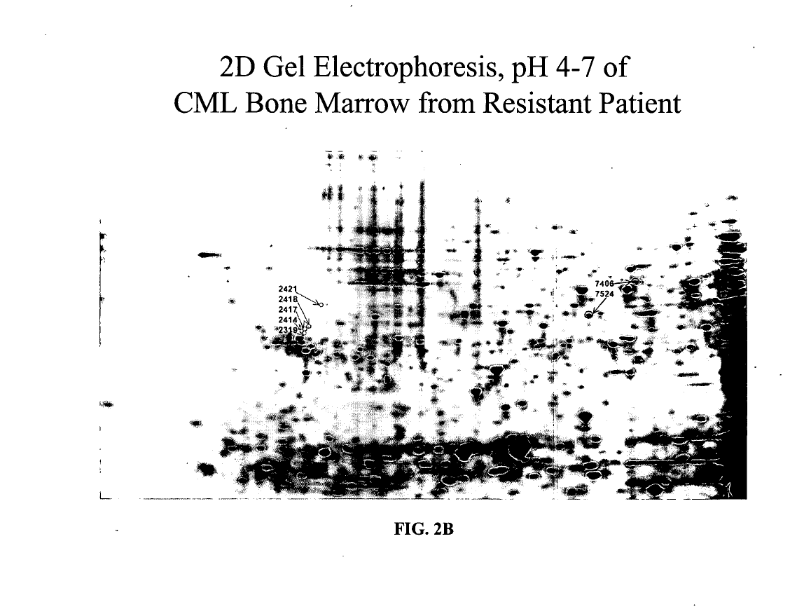

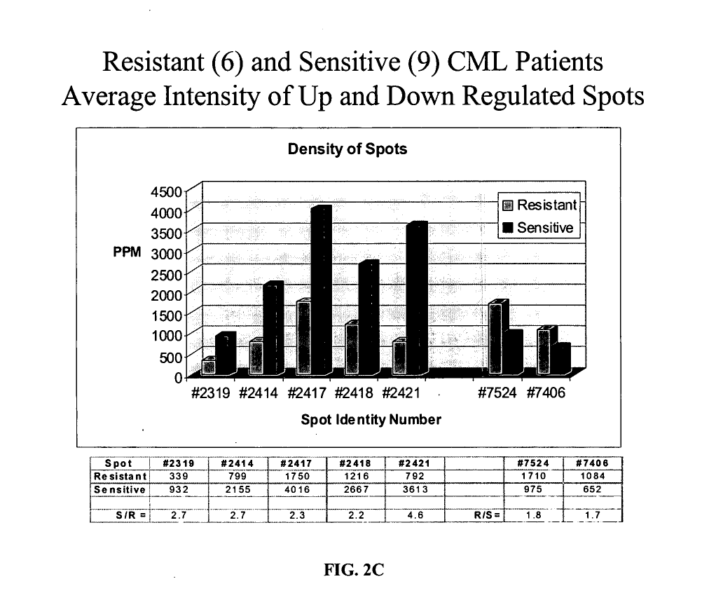

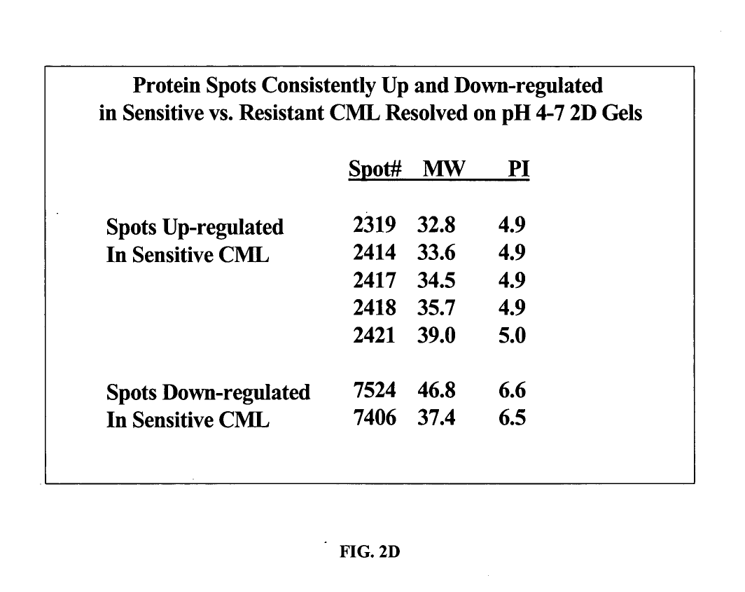

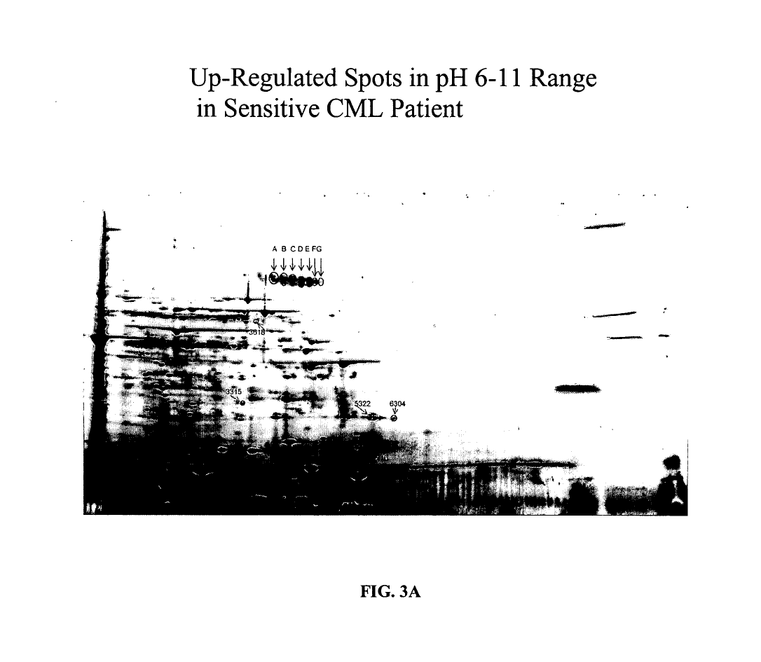

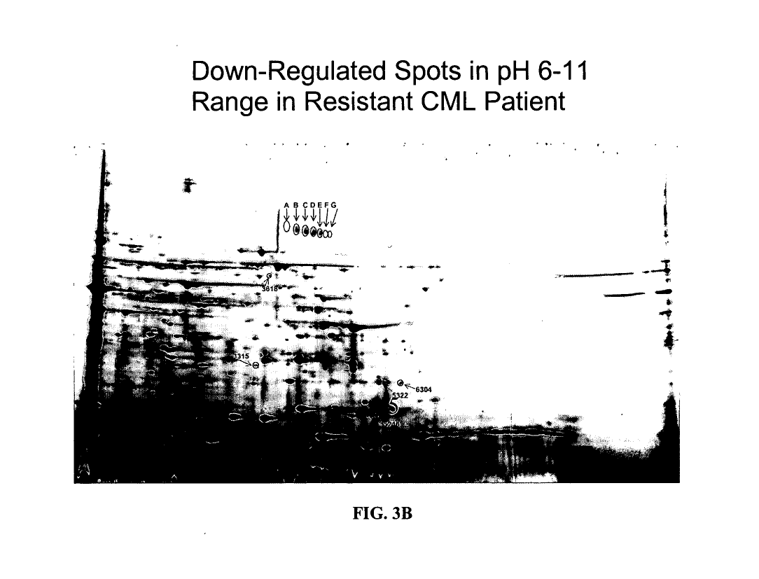

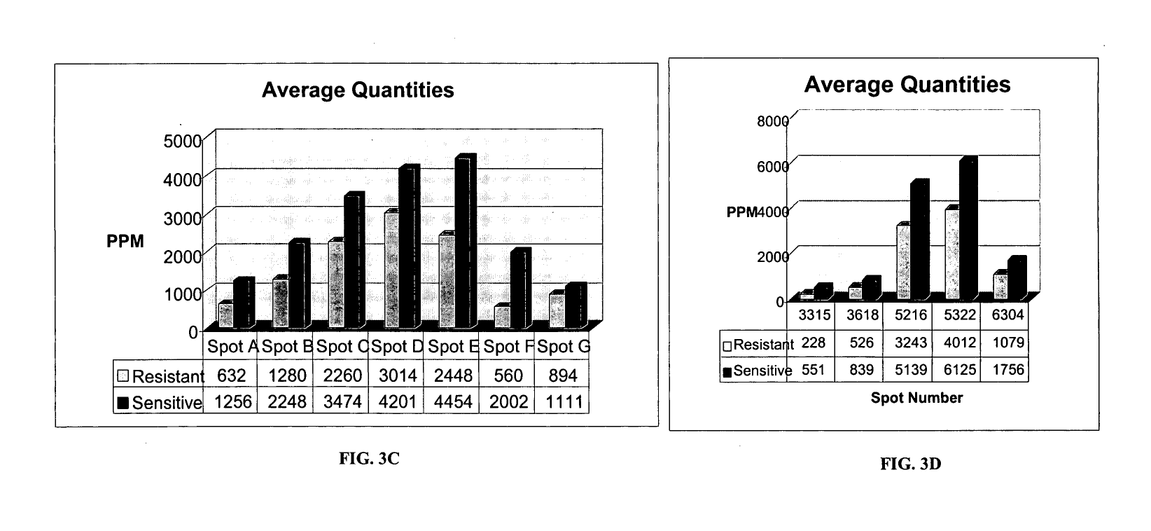

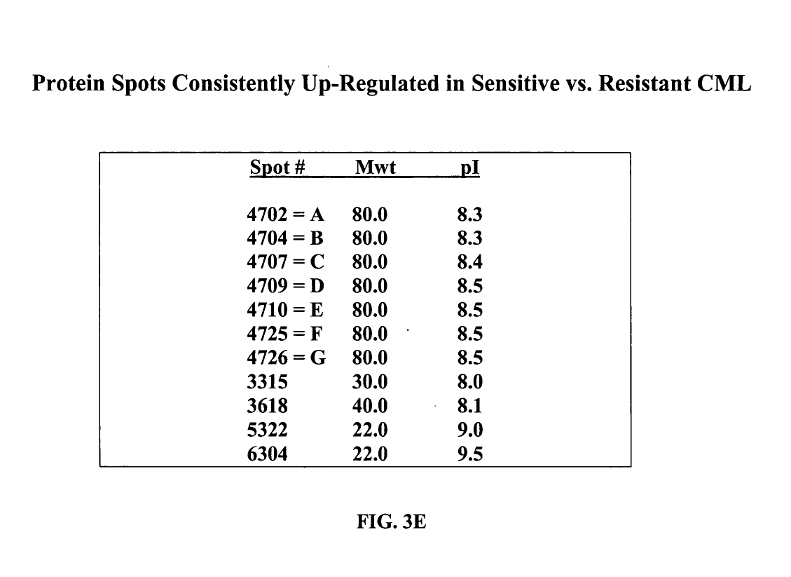

Results. A total of 19 spots were found to be differentially expressed between Gleevec-sensitive samples and Gleevec-resistant samples. In the pI 4-7 range, 5 spots were consistently up-regulated (spots 2319, 2414, 2417, 2418, and 2421) and 2 spots were consistently down-regulated (spots 7406 and 7524) in samples from Gleevec-sensitive patients relative to samples from Gleevec-resistant patients (FIGS. 2A, 2B, 2C, and 2D). In the pI 6-11 range, 12 spots were consistently up-regulated in the samples from the Gleevec-sensitive patients relative to samples from Gleevec-resistant patients (FIGS. 3A, 3B, 3C, and 3D). The differentially expressed spots were excised and the proteins identified, as described above. The proteins up-regulated in Gleevec-sensitive cells are listed in Table 4, and the proteins down-regulated in Gleevec-sensitive cells are listed in Table 5.

TABLE 4
Proteins Up-Regulated in Gleevec-Sensitive Cells

| | Proteins Up-Regulated in | Implications of Up-Regulation in |
|---|---|---|
| Spot | Gleevec-Sensitive Cells | Gleevec-Sensitive Cells |
| 2319 | DKFZp564L1878.1 | Cell differentiation activity: found to be |
| | [= Transmembrane protein | Down-regulated in hyperplastic colon |
| | with EGF-like and two | polyps, colorectal adenomas and |
| | follistatin-like domains 2; | carcinomas. Play a role in normal |
| | transmembrane protein | development of middle to late stages of |
| | TENB2; tomoregulin] | embryos and maintenance of adult central |
| | | nervous system tissues. May function as as |
| | | a ligand for erbB4- receptor, a regulator of |
| | | TGF-beta-related growth factor signaling by |
| | | direct interaction through the follistatin |
| | | modules, and a G-protein-coupled receptor |
| 2314 | Annexin A10 | Activation of Apoptosis: Down-regulation |
| | | of annexin A10 in hepatocellular carcinoma |
| | | is associated with vascular invasion, early |
| | | recurrence, and poor prognosis in synergy |
| | | with p53 mutation (Liu, Am J Pathol (2002)). |
| 2417 | P52rIPK protein homolog | Activation of Apoptosis: Unique form of |
| | DKFZp564L102.1 | P52rIPK, a growth suppressor and apoptotic |
| | | activator via up regulation of PKR and |
| | | PERK mediated eIF-2α phosphorylation. |
| | | P52rIPK accomplishes this by interacting |
| | | with P58IPK overcoming that protein's |
| | | interaction with and inhibition of PKR and |
| | | PERK mediated growth suppression and apoptosis. |
| 2418 | P52rIPK protein homolog | Activation of Apoptosis: Unique form of |
| | DKFZp564L102.1 | P52rIPK, a growth suppressor and apoptotic |
| | | activator via up regulation of PKR and |
| | | PERK mediated eIF-2α phosphorylation. |
| | | P52rIPK accomplishes this by interacting |
| | | with P58IPK overcoming that protein's |
| | | interaction with and inhibition of PKR and |
| | | PERK mediated growth suppression and apoptosis. |
| 2421 | Tumor necrosis factor | Activation of Apoptosis: Role of TRAF3 |
| | receptor superfamily member | and −6 in the Activation of the NF-kappa B |
| | XEDAR | and JNK Pathways by X-linked Ectodermal |
| | | Dysplasia Receptor (XEDAR) (Sinha, JBC (2002)). |
| A, 4702 | Many zinc finger proteins | Differentiation, regulation of cell growth, |
| B, 4704 | | stress response, apoptosis. |
| C, 4707 | | |
| D, 4709 | | |
| E, 4710 | | |
| F, 4725 | | |
| G, 4726 | | |
| 3315 | CD38 ADP-ribosyl cyclase 1 | Differentiation and Better Prognosis: |
| | | CD38 is an important prognostic factor and |
| | | reduced numbers may contribute to |
| | | leukemia escape from immune control |
| | | (Podesta, FASEB J (2002); Mohty, Br J |
| | | Haematol (2002)); Mainou-Fowler, Br J |
| | | Haematol (2002)) . . . |
| 3618 | Connective tissue growth | Activation of Apoptosis: CTGF (IGFBP- |
| | factor | rP2) is specifically expressed in malignant |
| | | lymphoblasts of patients with ALL and |
| | | CML. CTGF specifically binds IGFs with |
| | | low affinity and is considered to be a |
| | | member of the IGFBP superfamily (IGFBP- |
| | | rP2). Necrosis correlated with expression |
| | | of mRNA for tumor necrosis factor-alpha |
| | | (TNF-alpha), interleukin-10 (IL-10), matrix |
| | | metalloproteinase-9, and connective tissue |
| | | growth factor (CTGF) (Vorwerk, Br J |
| | | Cancer (2000)). |
| 5322 | CD28 (Tp44) | Activation of Apoptosis. |
| 6304 | BCL2-related ovarian killer | Activation of Apoptosis (Hsu PNAS 1997) |

TABLE 5
Proteins Down-Regulated in Gleevec Sensitive Cells

| | | Implications of |
|---|---|---|
| | Proteins Down-Regulated | Down-Regulation in |
| Spot | in Gleevec Sensitive Cells | Gleevec-Sensitive Cells |
| 7406 | Tumor necrosis factor | Anti-apoptotic Activity |
| | receptor superfamily member | (van Noesel Cancer |
| | 10D (Decoy receptor 2) | Res (2002)). |
| | (DcR2) (TRAIL-R4) | |
| 7524 | Chromosome 21 open | Unknown Function |
| | reading frame 63 (PRED34) | |
| | (SUE21) | |

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,683,202
U.S. Pat. No. 5,534,121
U.S. Pat. No. 5,928,906
U.S. Pat. No. 6,398,933
U.S. patent Application Ser. No. 10/301,512
Almendro et al., J. Immunol., 157(12):5411-5421, 1996.
Ausubel et al., In: Current Protocols in Molecular Biology, John, Wiley & Sons, Inc, New York, 1996.
Barber et al., Mol. Cell, Biol., 15(6):3138-31461995.
Barber et al., Proc. Natl. Acad. Sci. USA, 91(10):4278-4282, 1994.
Branford et al., Blood, 99:3472-3475, 2002.
Califano et al., Cancer Res., 56(11):2488-2492, 1996.
Carbonelli et al., FEMS Microbiol. Lett., 177(1):75-82, 1999.
Chandler et al., Proc. Natl. Acad. Sci. USA, 94(8):3596-601, 1997.
Cocea, Biotechniques, 23(5):814-816, 1997.
Druker et al., J. Clin. Invest., 105:3-7, 2000.
Druker et al., N. Engl. J. Med., 344:1038-1042, 2001.
Druker et al., Natl. Med., 2:561-566, 1996.
Fearon and Vogelstein, Cell, 61(5):759-767, 1990.
Freifelder, In: Physical Biochemistry Applications to Biochemistry and Molecular Biology, 2nd Ed. Wm. Freeman and Co., NY, 1982.
Gale et al., Biochemistry, 41(39):11878-11887, 2002.
Gale et al., Mol. Cell Biol., 18(2):859-871, 1998.
Gambacorti-Passerini et al., Blood, 100:367-368, 2002.
Gambacorti-Passerini et al., J. Natl. Cancer Inst., 92:1641-1650, 2000.
Goldman, Exp. Hemat., 32:2-10, 2004.
Gorre et al., Science, 293:876-880, 2001.
Hanash et al., Blood, 73(2):527-532, 1989.
Hanash et al., Proc. Natl. Acad. Sci. USA, 83(3):807-811, 1986.
Hochhaus et al., Leukemia, 6:2190-2196, 2002.
Hsu et al., Proc. Natl. Acad. Sci. USA, 94(23):12401-12406, 1997.
Koromilas et al., Science, 257(5077):1685-1689, 1992.
Kraus et al. FEBS Lett., 428(3):165-170, 1998.
Kuncewicz et al., Mol. Cell Proteomics, 2(3):156-163, 2003.
Lareyre et al., J. Biol. Chem., 274(12):8282-8290, 1999.
Le Coutre et al., Blood Cells Mol. Dis., 28:75-85, 2002.
Lee et al., Science, 13:235(4794):1394-1399, 1987.
Levenson et al., Hum. Gene Ther., 9(8):1233-1236, 1998.
Liu et al., Am. J. Pathol., 160(5):1831-1837, 2002.
Macejak and Sarnow, Nature, 353:90-94, 1991.
Mahon et al., Blood, 101:2368-2373, 2003.
Mainou-Fowler et al., Br. J. Haematol., 118(3):755-761, 2002.
Maniatis, et al., Molecular Cloning, A Laboratory Manual, Cold Spring Harbor Press, Cold Spring Harbor, N.Y., 1990.
Meurs et al., Proc. Natl. Acad. Sci., USA, 90(1):232-236, 1993.
Mohty et al., Br. J. Haematol., 119(1):115-118, 2002.
Nomoto et al., Gene, 236(2):259-271, 1999.
Pelletier and Sonenberg, Nature, 334(6180):320-325, 1988.
Podesta et al., FASEB J., 17(2):310-312, 2002.
Sambrook et al., In: Molecular cloning, Cold Spring Harbor Laboratory Press, Cold Spring Harbor, N.Y., 2001.
Sawyers et al., Blood, 99:3530-3539, 2002.
Shah et al., Cancer Cell, 2:117-125, 2002.
Sinha et al., J. Biol. Chem., 277(47):44953-44961, 2002.
Talpaz et al., Blood, 99:1928-1937, 2002.
Tan et al., Mol. Cell Biol., 18(5):2431-2443, 1998.
Tsumaki et al., J. Biol. Chem., 273(36):22861-22864, 1998.
Van Noesel et al., Cancer Res., 62(7):2157-2161, 2002.
Vorwerk et al., Br. J. Cancer, 83(6):756-760, 2000.
Voss et al., Int. J. Cancer, 91(2):180-186, 2001.
Wu et al., Biochem. Biophys. Res. Commun., 233(1):221-2266, 1997.
Yan et al., Proc. Natl. Acad. Sci. USA, 99(25):15920-15925, 2002.
Zhao-Emonet et al., Biochim. Biophys. Acta, 1442(2-3):109-119, 1998.

1-9. (canceled)

10. A method for predicting a subject's sensitivity or resistance to an Abl kinase inhibitor comprising:
(a) obtaining a sample from the subject;
(b) determining a protein expression profile for the subject; and
(c) comparing the subject's protein expression profile with a reference protein expression profile to predict the subject's sensitivity or resistance to an Abl kinase inhibitor.

11. The method of claim 10, wherein the Abl kinase inhibitor is imatinib mesylate.

12. The method of claim 10, wherein the sample is a cell or a composition of cells.

13. The method of claim 10, wherein the sample is a bone marrow sample, a peripheral blood sample, or a tumor sample.

14. The method of claim 10, wherein the protein expression profile is determined by evaluating transcription levels.

15. The method of claim 10, wherein the protein expression profile is determined by evaluating protein levels.

16. The method of claim 15, wherein evaluating the protein levels involves performing two-dimensional gel electrophoresis.

17. The method of claim 14, wherein evaluating the transcription levels involves performing RT-PCR.

18. The method of claim 10, wherein the protein expression profile comprises one or more protein markers.

19. The method of claim 10, wherein the protein expression profile comprises one or more of the proteins in Table 1.

20-23. (canceled)

24. The method of claim 10, wherein the subject has a hematologic malignancy.

25. The method of claim 24, wherein the hematologic malignancy is leukemia, non-Hodgkin lymphoma, Hodgkin lymphoma, myeloma, or myelodysplastic syndrome.

26. The method of claim 25, wherein the leukemia is acute myelogenous leukemia, chronic myelogenous leukemia, acute lymphocytic leukemia, or chronic lymphocytic leukemia.

27-46. (canceled)

47. A method for identifying a compound that inhibits P58IPK interaction with PKR comprising:
(a) obtaining a compound that is a candidate inhibitor of the interaction between P58IPK and PKR;
(b) combining the compound with P58IPK and PKR; and
(c) assessing whether the compound inhibits interaction between P58IPK and PKR to identify a compound that inhibits P58IPK interaction with PKR.

48. The method of claim 47, further comprising assessing the interaction between P58IPK and PKR in the absence of the compound.

49. The method of claim 47, wherein combining the compound with P58IPK and PKR occurs in a cell.

50. The method of claim 47, further comprising manufacturing a pharmaceutical composition comprising the compound.

51-54. (canceled)

55. The method of claim 49, wherein the cancer cell is a leukemia cell.

56. (canceled)

57. A method for inhibiting the growth of a cancer cell comprising contacting the cancer cell with an expression construct comprising a polynucleotide encoding P52rIPK or P52rIPK homolog DKFZp564B102.1.

58. The method of claim 57, further comprising contacting the cancer cell with IFN-γ.

59. The method of claim 57, further comprising contacting the cancer cell with imatinib mesylate.

60. The method of claim 57, wherein the cancer cell is an imatinib mesylate resistant leukemia cell.

61-62. (canceled)

63. The method of claim 49, further comprising evaluating the expression of one or more of P52rIPK or P52rIPK homolog DKFZp564B 102.1 in the cell in the presence of the candidate compound.

64-65. (canceled)

66. The method of claim 10, wherein the reference protein expression profile is obtained by a method comprising:
(a) obtaining a first cell, wherein the first cell is sensitive to the Abl kinase inhibitor;
(b) obtaining a second cell, wherein the second cell is resistant to the Abl kinase inhibitor; and
(c) identifying a protein, a group of proteins, or a protein pattern that is differentially expressed between the first cell and the second cell,
wherein the differentially expressed protein, group of proteins, or protein pattern associated with sensitivity or resistance to the Abl kinase inhibitor is the reference protein expression profile.

67. The method of claim 15, wherein evaluating the protein levels involves performing mass spectrometry.

68. The method of claim 67, wherein the mass spectrometry is MALDI-TOF MS, SELDI-TOF MS, or MS-MS.
